(19) United States
(12) Patent Application Publication
(10) Pub. No.: US 2017/0349919 A1
(43) Pub. Date: Dec. 7, 2017

(54) MICRO-ORGANISM FOR THE PRODUCTION OF STEREO-SPECIFIC S, S-2,3-BUTANEDIOL

(71) Applicant: DANMARKS TEKNISKE UNIVERSITET, Kgs. Lyngby (DK)

(72) Inventors: Christian Solem, Naerum (DK); Peter Ruhdal Jensen, Gentofte (DK); Jun Chen, Copenhagen NV (DK); Jianming Liu, Virum (DK)

(21) Appl. No.: 15/536,470

(22) PCT Filed: Dec. 18, 2015
(86) PCT No.: PCT/EP2015/080446

§ 371 Date: Jun. 15, 2017

(30) Foreign Application Priority Data
Dec. 19, 2014 (EP) 14199353.5
Oct. 27, 2015 (EP) 15191703.6

(51) Int. Cl. C12P 7/18; C12N 9/02

(52) U.S. Cl. CPC C12P 7/18; C12Y 101/01304; C12N 9/0036; C12Y 101/01076

(57) ABSTRACT

The invention relates to a genetically modified lactic acid bacterium capable of producing (S,S)-2,3-butanediol stereo specifically from glucose under aerobic conditions. Additionally the invention relates to a method for producing (S,S)-2,3-butanediol and L-acetoin using the genetically modified lactic acid bacterium, under aerobic conditions in the presence of a source of iron-containing porphyrin or a source of metal ions (Fe³⁺/Fe²⁺). The lactic acid bacterium is genetically modified to express heterologous genes encoding enzymes catalysing the stereo-specific synthesis of (S,S)-2,3-butandiol; and additionally a number of genes are deleted in order to maximise the production of (S,S)-2,3-butanediol as compared to other products of oxidative fermentation.

### FIELD OF THE INVENTION

The present invention provides a genetically modified lactic acid bacterium capable of producing (S,S)-2,3-butanediol stereo specifically from glucose under aerobic conditions. Additionally the invention provides a method for producing (S,S)-2,3-butanediol using the genetically modified lactic acid bacterium, under aerobic conditions in the presence of a source of iron-containing porphyrin or a source of metal ions (Fe³⁺/Fe²⁺). The lactic acid bacterium is genetically modified to express heterologous genes encoding a meso-2,3-butanediol dehydrogenase (E.C. 1.1.1.-), having diacetyl reductase ((S)-acetoin forming; E.C. 1.1.1.5/1.1.1.304) activity, and a L-butanediol dehydrogenase (E.C. 1.1.1.76). Additionally genes encoding polypeptides having lactate dehydrogenase (E.C 1.1.1.27/E.C.1.1.1.28); α-acetolactate decarboxylase (E.C 4.1.1.5); diacetyl reductase ((R)-acetoin forming; EC. 1.1.1.303); acetoin reductase (EC. 1.1.1.5); butanediol dehydrogenase ((R,R)-butane-2,3-diol forming; E.C. 1.1.1.4/1.1.1.-); a water-forming NADH oxidase (E.C. 1.6.3.4); and optionally phosphotransacetylase (E.C. 2.3.1.8) and alcohol dehydrogenase (E.C. 1.2.1.10); are deleted from the bacterium.

### BACKGROUND OF THE INVENTION

2,3-butanediol (2,3-BDO) is a high value commodity chemical, usually produced petrochemically from oil. 2,3-butanediol, also known as 2,3-butylene glycol, dimethylene glycol, or dimethylethylene glycol, has many current and potential applications, including: plasticizers, aviation fuel, printing inks, perfumes, fumigants, spandex, and as a carrier for pharmaceuticals (Celinska & Grajek, 2009).

2,3-butanediol exists in 3 isomeric forms: L-(+)-2,3-butanediol (S,S)-; D-(−)-2,3-butanediol (R,R)-; and meso-2,3-butanediol. The chiral forms of 2,3-BDO, in particular L-(+)-2,3-butanediol (S,S)-, have additional value in the provision of chiral groups required in the synthesis of drugs and liquid crystals.

The use of the synthetic machinery in microorganisms for the production of organic chemicals is desirable since it allows for synthesis of relatively complex compounds, such as 2,3-BDO or isomers thereof, while avoiding the harsh conditions associated with organic chemical synthesis, and often provides the added advantage of an improved yield and purity of the product.

2,3-butanediol is synthesized by two different pathways in micro-organisms, in both cases from α-acetolactate. α-Acetolactate can be converted into one isomer of acetoin, namely D-acetoin, by α-acetolactate decarboxylase and D-acetoin can then be converted into either meso- or (R, R)-2,3-butanediol depending on the properties of BDH that is present. In the presence of oxygen, however, α-acetolactate is unstable and can be converted into diacetyl that can also be enzymatically reduced into L-acetoin and further to L-(+)-2,3-butanediol. Bacterial strains differ as to which stereoisomers of 2,3-butanediol is formed, dependent on the stereo-specificity of the expressed 2,3-butanediol dehydrogenases. Three 2,3-BD dehydrogenases are proposed to exist: meso-2,3-BD dehydrogenase (D-(−)-acetoin forming), meso-2,3-BD dehydrogenase (L-(+)-acetoin forming), and L-(+)-2,3-BD dehydrogenase (Ui et al., 1984, Ji et al., 2011).

The microbial production of 2,3-butanediol (2,3-BDO) and its isomers requires a suitable host micro-organism. One option is to select a natural producer, but unfortunately the most efficient 2,3-BDO producers (Klebsiella pneumonia, Klebsiella oxytoca, Enterobacter aerogenes, Serratia marcescens as well as an engineered stain of Escherichia coli), are all categorized by the World Health Organization (WHO) as risk group 2 species (pathogenic), which makes their use in the large-scale production of 2,3-BDO particularly challenging and costly (Biswas et al., 2012). Another decisive factor in selecting a host micro-organism, is to identify a host that can produce 2,3-BDO, and/or isomers thereof, from inexpensive renewable raw materials via an efficient bio-conversion process.

Ui et al (2004) describe genetically engineered E. coli capable of converting diacetyl to L-(+)-2,3-butanediol (S,S-2,3-BDO), as the main chiral form, by the simultaneous expression of a meso-2,3-butandiol dehydrogenase gene having diacetyl reductase activity, encoded by the BudC from Klebsiella pneumonia IAM 1063; and a L-(+)2,3-butanediol dehydrogenase gene derived from Brevibacterium saccharolyticum. However, its use in the production of S,S-2,3-BDO requires a supply of the substrate diacetyl.

Members of the Lactic acid bacteria genus provide a safer alternative, since they are included in the FDA GRAS list. Members, such as Lactococcus lactis (L. lactis), are potentially efficient bio-convertors, since they are able to channel >90% of metabolized sugar into fermentation products (Thomas, 1976), at a higher rate than other established production organisms. Furthermore, although some L. lactis strains, especially the dairy isolates, are quite fastidious, they are able to grow in cheap media based on whey or condensed corn soluble, a fuel ethanol production byproduct (Wolf-Hall et al., 2009).

Lactococcus lactis (L. lactis) has genes encoding the enzymes needed for making 2,3-butanediol, but most strains do not normally produce 2,3-butanediol, and in exceptional cases (L. lactis subsp. lactis biovar. diacetylactis strains) only in minor amounts when grown in the presence of citrate (Crow, 1990). In order to take advantages of lactic acid bacteria as a safe host for production of (S,S)-2,3-butanediol, there is a need for developing strains of lactic acid bacteria that are capable of stereo-specifically producing (S,S)-2,3-butanediol in improved yields from cheap raw materials.

### SUMMARY OF THE INVENTION

The invention provides a genetically modified lactic acid bacterium for production of S,S-2,3-butanediol, wherein said microorganism comprises:
a) one transgene encoding a polypeptide, wherein the polypeptide has an enzymatic activity of both a diacetyl reductase (E.C.1.1.1.304) and a L-butanediol dehydrogenase (E.C. 1.1.1.76); or
   two transgenes encoding two polypeptides, wherein one polypeptide has an enzymatic activity of a diacetyl reductase (E.C.1.1.1.304) and the second polypeptide has an enzymatic activity of a L-butanediol dehydrogenase (E.C. 1.1.1.76);
   and whereby expression of said one or two transgenes in said microorganism confers the capability to convert diacetyl to S,S-2,3-butanediol;
   and wherein the genome of said lactic acid bacterium is deleted for genes or lacks genes encoding polypeptides having an enzymatic activity of:
b) lactate dehydrogenase (E.C 1.1.1.27 or E.C.1.1.1.28),
c) α-acetolactate decarboxylase (E.C 4.1.1.5)
d) diacetyl reductase (E.C.1.1.1.303)
e) butanediol dehydrogenase (E.C. 1.1.1.4)
f) acetoin reductase (EC:1.1.1.5) and
g) NADH oxidase (E.C. 1.6.3.4).

In a further embodiment, the genome of said genetically modified lactic acid bacterium is additionally deleted for genes encoding polypeptides having an enzymatic activity of:
h) a phosphotransacetylase (E.C.2.3.1.8) and
i) an alcohol dehydrogenase (E.C. 1.2.1.10).

According to further embodiment, the genetically modified lactic acid bacterium comprises one transgene encoding one polypeptide, wherein the polypeptide has an enzymatic activity of a diacetyl reductase (E.C.1.1.1.304) and a L-butanediol dehydrogenase (E.C. 1.1.1.76) and is capable of converting diacetyl to S,S-2,3-butanediol.

In a further embodiment, the genetically modified lactic acid bacterium belongs to a genus selected from the group consisting of Lactococcus, Lactobacillus, Pediococcus, Leuconostoc, Streptococcus, Oenococcus, and Bacillus.

The invention also provides a method for the production of S,S-2,3-butanediol, comprising the steps of: a) introducing the genetically modified lactic acid bacterium of the invention into a growth medium to produce a culture, b) providing a source of protoporphyrin IX and/or iron-containing porphyrin or providing a source of Fe³⁺ ions, c) providing aerobic culture conditions, d) recovering S,S-2,3-butanediol produced by said culture, and optionally e) isolating the recovered S,S-2,3-butanediol.

The invention also includes the use of the genetically modified lactic acid bacterium of the invention for production of S,S-2,3-butanediol and additionally L-acetoin.

### Abbreviations and Terms

gi number: (genInfo identifier) is a unique integer which identifies a particular sequence, independent of the database source, which is assigned by NCBI to all sequences processed into Entrez, including nucleotide sequences from DDBJ/EMBL/GenBank, protein sequences from SWISS-PROT, PIR and many others.

Amino acid sequence identity: The term “sequence identity” as used herein, indicates a quantitative measure of the degree of homology between two amino acid sequences of substantially equal length. The two sequences to be compared must be aligned to give a best possible fit, by means of the insertion of gaps or alternatively, truncation at the ends of the protein sequences. The sequence identity can be calculated as ((Nref-Ndif)100)/(Nref), wherein Ndif is the total number of non-identical residues in the two sequences when aligned and wherein Nref is the number of residues in one of the sequences. Hence, the peptide sequence AGTCAGTC will have a sequence identity of 75% with the sequence AATCAATC (Ndif=2 and Nref=8). A gap is counted as non-identity of the specific residue(s), i.e. the peptide sequence AGTGTC will have a sequence identity of 75% with the peptide sequence AGTCAGTC (Ndif=2 and Nref=8). Sequence identity can alternatively be calculated by the BLAST program e.g. the BLASTP program (Pearson W. R and D. J. Lipman (1988)) (www.ncbi.nlm.nih.gov/cgi-bin/BLAST). In one embodiment of the invention, alignment is performed with the sequence alignment method ClustalW with default parameters as described by Thompson J., et al 1994, available at http://www2.ebi.ac.uk/clustalw/.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, and Glutamine.

Deleted gene: the deletion of a gene from the genome of a microbial cell leads to a loss of function of the gene and hence where the gene encodes a polypeptide the deletion results in a loss of expression of the encoded polypeptide. Where the encoded polypeptide is an enzyme, the gene deletion leads to a loss of detectable enzymatic activity of the respect polypeptide in the microbial cell.

Native gene: endogenous gene in a microbial cell genome, homologous to host micro-organism.

### DESCRIPTION OF THE FIGURES

FIG. 1 Cartoon showing the modifications of the metabolic pathway of a lactic acid bacterium for the stereo-specific production of (S,S)-2,3-butanediol.

FIG. 2 HPLC product profile of strain CS4701 derived from Lactococcus lactis subsp. cremoris was grown under aerobic conditions in M17 medium supplemented with 2% glucose as carbon source, and hemin supplied at a concentration of 5 μg/ml (A); 1 μg/ml (B); 0.5 μg/ml (C) and 0.1 μg/ml (D). The (S,S)-2,3-butanediol peak (highlighted) is detected at 17.3 minutes.

FIG. 3 HPLC diagrams of butanediol isomers detected in a fermentation sample obtained from a cell culture of CS4701m derived from Lactococcus lactis subsp. cremoris grown under aerobic conditions in M17 medium supplemented with 60 mM glucose (10.8. g/l glucose) as carbon source, and 10 mM Fe³⁺. Upper and middle panels shows HPLC diagrams of 10 mM meso-2,3-butandiol and 10 mM (S,S)-2,3-butandiol respectively run as standards; and the lower panel is the HPLC diagram of the fermentation sample.

FIG. 4 Cartoon showing that stereo-specific production of (S,S)-2,3-butanediol in the genetically modified L. lactis strain of the invention, expressing a diacetyl-insensitive L-butanediol dehydrogenase, is characterized by a balanced redox due to re-cycling of NADH produced in glycolysis by the reduction reactions in the synthesis of (S,S)-2,3-butanediol.

### DETAILED DESCRIPTION

### I A Lactic Acid Bacterium for Production of (S,S)-2,3-butanediol

The present invention provides a genetically modified lactic acid bacterium capable of producing (S,S)-2,3-butanediol stereo specifically from glucose under aerobic conditions. According to a first embodiment, the bacterium of the invention comprises two transgenes: (1) encoding a meso-2,3-butanediol dehydrogenase (E.C. 1.1.1.-), having strong diacetyl reductase activity (E.C.1.1.1.5/1.1.1.304) for converting diacetyl (DA) to L-acetoin (L-AC); and (2) a L-butanediol dehydrogenase (E.C.1.1.1.76) converting L-AC to L-BD. Although L-butanediol dehydrogenases are known to convert DA to L-AC, this activity is very weak, and furthermore DA is known to act as a competitive inhibitor of the reduction of L-AC. Hence, the efficient conversion of diacetyl (DA) to L-BD requires a two-step reaction catalyzed by these two enzymes.

The bacterium of the invention is also characterized by inactivation of a number of metabolic pathways to enhance metabolic flux from pyruvate for diacetyl, which is the precursor in the pathway for stereospecific synthesis of (S,S)-2,3-butanediol.

The bacterium of the invention is also characterized by inactivation of the endogenous 2,3-butanediol synthesis pathway.

The bacterium of the invention is also characterized by an inactivated water forming cytoplasmic NADH oxidase in order to maintain and balance NADH reducing capacity required for reductive synthesis of (S,S)-2,3-butanediol.

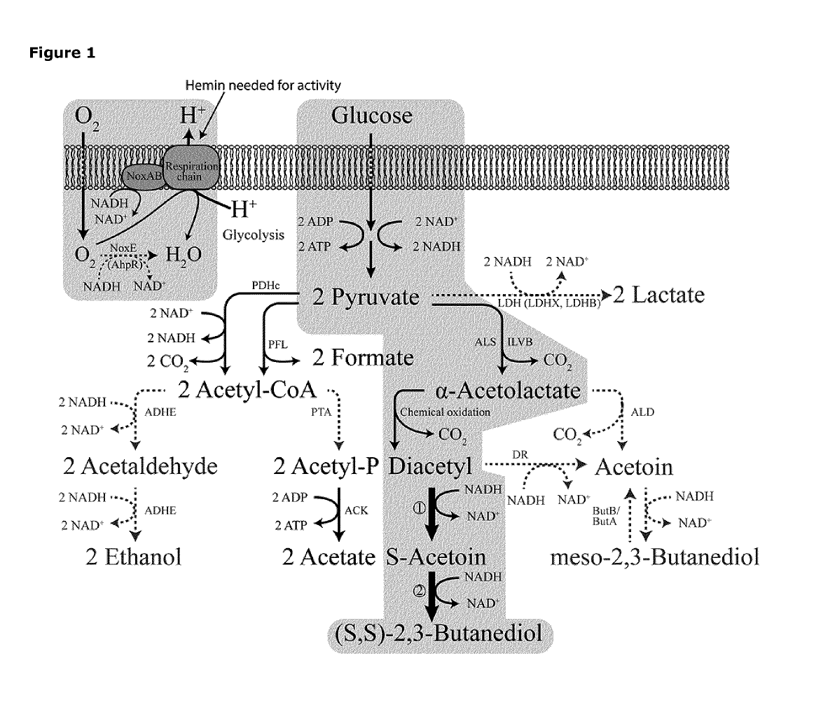

The production of (S,S)-2,3-butanediol by the lactic acid bacterium of the invention under aerobic conditions, is thought to be mediated via a synthetic pathway illustrated in FIG. 1, including the synthetic intermediates: pyruvate, α-acetolactate, diacetyl, and L-acetoin. The conversion of α-acetolactate into diacetyl occurs by chemical oxidation, provided that the lactic acid bacterium of the invention is maintained under aerobic conditions. Diacetyl, derived from oxidation of α-acetolactate, is the substrate for the stereo-specific production of (S,S)-2,3-butanediol via L-acetoin catalyzed by meso-2,3-butanediol dehydrogenase (E.C. 1.1.1.-) and a L-butanediol dehydrogenase (E.C. 1.1.1.76).

Production of (S,S)-2,3-butanediol by the lactic acid bacterium according to the first embodiment of the invention requires specific cultivation conditions. While not being bound by theory, it is hypothesized that the aerobic conditions required for chemical oxidation of α-acetolactate, activates cellular NADH oxidase activity depriving the cell of sufficient NADH reducing power needed for (S,S)-2,3-butanediol. While inactivation of the water forming cytoplasmic NADH oxidase in the lactic acid bacterium of the invention prevents the depletion of cellular NADH levels under aerobic growth conditions, the cells are then unable to chemically oxidize α-acetolactate. Surprisingly, the provision of a supply of iron-containing porphyrin, in limited amounts, is shown to be essential for the lactic acid bacterium according to the first embodiment of the invention to grow and facilitates its production of (S,S)-2,3-butanediol.

The features of the genetically modified lactic acid bacterium, according to the first embodiment of the invention, are described in further detail below.

### Ii Transgenic Expression of an Enzyme Having Diacetyl Reductase Activity

The bacterium of the invention expresses a polypeptide having diacetyl reductase (DAR) activity (E.C:1.1.1.5/1.1.1.304) that converts diacetyl (DA) to L-acetoin (L-AC). The amino acid sequence of the polypeptide has at least 70, 72, 74, 76, 78, 80, 82, 84, 86, 88, 90, 92, 94, 96, 98, 99 or 100% sequence identity to the amino acid sequence of the meso-2,3-butanediol dehydrogenase encoded by the Klebsiella pneumonia budC gene (SEQ ID NO: 2). Alternatively, the polypeptide having diacetyl reductase (DAR) activity (E.C:1.1.1.5/1.1.1.304), has the amino acid sequence selected from among SEQ ID NO: 4 (derived from Enterobacter aerogenes KCTC 2190); SEQ ID NO: 6 (derived from Serratia sp. ATCC 39006); SEQ ID NO: 8 (derived from Pluralibacter gergoviae strain FB2); and SEQ ID NO: 10 (derived from Kosakonia sacchari SP1).

### Iii Transgenic Expression of an Enzyme Having L-butanediol Dehydrogenase Activity

A polypeptide having L-butanediol dehydrogenase (E.C. 1.1.1.76) converting L-acetoin (L-AC) to (S,S)-2,3-butanediol, comprises an amino acid sequence having at least 70, 72, 74, 76, 78, 80, 82, 84, 86, 88, 90, 92, 94, 96, 98, 99 or 100% sequence identity to the amino acid sequence of the L-(+)-2,3-butanediol dehydrogenase (SEQ ID NO:12) derived from Brevibacterium saccharolyticum budC gene. For example, the polypeptide having L-(+)-2,3-butanediol dehydrogenase activity may be selected from a polypeptide having amino acid sequence SEQ ID NO:14 (derived from Corynebacterium glutamicum); or SEQ ID NO: 16 (derived from Microbacterium paraoxydans).

One or both of the DAR and L-BDH enzymatic activities and their corresponding enzymatic structural domains (as described above) may be present in individual proteins, each encoded by a gene, or one or both of the enzymatic activities may be present in a fusion protein, where the fusion protein comprises at least both active enzymatic structural domains encoded by a gene. The gene in the micro-organism of the invention that expresses a polypeptide having one or both active DAR and L-BDH enzymatic domains, may be a transgene that is adapted for expression in the selected host cell, by employing a codon usage optimized for the given lactic acid bacterial cell, such codon optimization being well-known in the art. Nucleic acid molecules encoding a polypeptide having one or more enzymatic domain can be synthesized chemically, where the nucleic acid sequence of the molecule is selected to provide the codon usage optimized for the given host cell. The nucleic acid sequence of DNA molecules encoding the respective DAR and L-BDH enzymatic activities of the (S,S)-2,3-butanediol pathway are exemplified in the sequence listing.

In some embodiments, one or more of the genes encoding one or more polypeptide having an enzymatic activity associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA, although RNA vectors are also available. Vectors include, but are not limited to: plasmids, fosmids, phagemids, virus genomes and artificial chromosomes. A suitable vector includes one which is able to replicate autonomously (self-replicating vector) or is integrated (integration vector) in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium; or may occur just a single time per host before the host reproduces by mitosis. In the case of phage, replication may occur actively during a lytic phase or passively during a lysogenic phase.

When the one or more genes encoding one or more polypeptide having DAR and L-BDH enzymatic activity required for (S,S)-2,3-butanediol synthesis are expressed in a micro-organism of the invention, a variety of transcription control sequences (e.g., promoter/enhancer sequences) may be operably joined to the coding sequence encoding the respective polypeptide, such as to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Methods for introducing one or more transgene encoding the encoding polypeptides having one or more enzymatic activities into a host micro-organism of the invention is described in section III.

### Iiii Endogenous Genes Deleted to Enhance Metabolic Flux from Pyruvate to Diacetyl

The lactic acid bacterium of the invention is adapted to produce (S,S)-2,3-butanediol from glucose under aerobic conditions. The lactic acid bacterium of the invention is characterised by an enhanced metabolic flux from pyruvate to diacetyl, due to reduced activity in the enzymes in the pathways leading to the synthesis of lactate, acetate and ethanol. Deletion of genes encoding enzymes of the lactate pathway in the lactic acid bacterium reduces the metabolic flux towards lactate. The production of acetate and ethanol by the lactic acid bacterium of the invention is reduced when the bacterium is cultivated under aerobic conditions, in a defined growth medium lacking lipoic acid. When the bacterium is cultivated under aerobic conditions, this inactivates the enzyme pyruvate formate lyase that forms formate and acetyl-CoA, which are the precursors of the acetate and ethanol pathways. Since the enzyme, pyruvate dehydrogenase, requires lipoic acid for activity, the use of a lipoic acid-deficient growth medium (supplemented with acetate) inactivates the synthesis of acetyl-CoA by pyruvate dehydrogenase and the down-stream production of acetate and ethanol. When the lactic acid bacterium of the invention is grown under anaerobic conditions in a minimal medium deficient in lipoic acid, the requirement for acetyl-CoA is met by adding acetate to the growth medium.

In another embodiment, the metabolic flux towards lactate, acetate and ethanol in the lactic acid bacterium of the invention is reduced by deletion of one or more genes encoding enzymes of both the lactate, acetate and ethanol pathways.

Deletion of the lactate pathway: The lactic acid bacterium of the invention is characterised by knockouts of one or more endogenous native gene encoding a polypeptide having lactate dehydrogenase activity causing a block in the lactate synthesis pathway in the bacterium. Deletion of at least one gene (e.g. ldh) encoding a lactate dehydrogenase enzyme (E.C 1.1.1.27 or E.C.1.1.1.28) provides a lactic acid bacterium of the invention that is depleted in lactate production. For example, where the lactic acid bacterium of the invention belongs to a given genus, the deleted endogenous gene is one encoding a polypeptide having lactate dehydrogenase activity in that genus. Preferably the polypeptide having lactate dehydrogenase activity (E.C 1.1.1.27 or E.C.1.1.1.28) has at least 70, 72, 74, 76, 78, 80, 82, 84, 86, 88, 90, 92, 94, 96, 98, 99 or 100% amino acid sequence identity to one of the following sequences: SEQ ID NO: 18 in a Lactococcus species (e.g. Lactococcus lactis); SEQ ID NO: 20, 22, or 24 in a Lactobacillus species (e.g. Lactobacillus acidophilus); SEQ ID NO: 26 in a Lactobacillus species (e.g. Lactobacillus delbrueckii); SEQ ID NO. 28, 30 or 32 in a Lactobacillus species (e.g. Lactobacillus casei), SEQ ID NO. 34 or 36 in a Lactobacillus species (e.g. Lactobacillus plantarum); SEQ ID NO: 38 in a Pediococcus species (e.g. Pediococcus pentosaceus), SEQ ID NO: 40 or 42 in a Leuconostoc species (e.g. Leuconostoc mesenteroides), SEQ ID NO: 44 in a Streptococcus species (e.g. Streptococcus thermophilus), SEQ ID NO: 46 or 48 in a Oenococcus species (e.g. Oenococcus oeni), and SEQ ID NO: 50 or 52 in a Bacillus species (e.g. Bacillus coagulans).

In one embodiment, an additional endogenous gene, encoding a polypeptide having lactate dehydrogenase enzymatic activity (E.C 1.1.1.27 or E.C.1.1.1.28), is deleted from the lactic acid bacterium of the invention. For example, where the lactic acid bacterium of the invention belongs to the genus Lactococcus, the deleted gene (IdhX) encodes a polypeptide having at least 70, 72, 74, 76, 78, 80, 82, 84, 86, 88, 90, 92, 94, 96, 98, 99 or 100% amino acid sequence identity to SEQ ID NO: 54.

In one embodiment, an additional endogenous gene, encoding a polypeptide having lactate dehydrogenase enzymatic activity (E.C 1.1.1.27 or E.C.1.1.1.28), is deleted from the lactic acid bacterium of the invention. For example, where the lactic acid bacterium of the invention belongs to the genus Lactococcus, the deleted gene (ldhB) encodes a polypeptide having at least 70, 72, 74, 76, 78, 80, 82, 84, 86, 88, 90, 92, 94, 96, 98, 99 or 100% amino acid sequence identity to SEQ ID NO: 56. Further, where the lactic acid bacterium of the invention belongs to the genus Lactococcus, the three genes (Idh, IdhB and IdhX) encoding a polypeptide having at least 70% amino acid sequence identity to SEQ ID NO: 18, 54 and 56 respectively may be deleted.

Deletion of the acetate pathway: In one embodiment, the lactic acid bacterium of the invention is characterised by knockout of the endogenous native gene encoding a phosphotransacetylase (E.C.2.3.1.8), causing a block in the acetate synthesis pathway in the bacterium. Deletion of a gene (e.g. pta) encoding a phosphotransacetylase enzyme provides a lactic acid bacterium of the invention that is blocked in acetate production. For example, where the lactic acid bacterium of the invention belongs to a given genus, the deleted endogenous gene is one encoding a polypeptide having phosphotransacetylase activity (E.C.2.3.1.8) in that genus. Preferably the polypeptide having phosphotransacetylase activity has at least 70, 72, 74, 76, 78, 80, 82, 84, 86, 88, 90, 92, 94, 96, 98, 99 or 100% amino acid sequence identity to one of the following sequences: SEQ ID NO: 58 in a Lactococcus species (e.g. Lactococcus lactis); SEQ ID NO: 60, 62, 64, and 66 in a Lactobacillus species (e.g. Lactobacillus acidophilus, Lactobacillus delbrueckii, Lactobacillus casei, Lactobacillus plantarum), SEQ ID NO: 68 in a Pediococcus species (e.g. Pediococcus pentosaceus), SEQ ID NO: 70 in a Leuconostoc species (e.g. Leuconostoc mesenteroides), SEQ ID NO: 72 in a Streptococcus species (e.g. Streptococcus thermophilus), SEQ ID NO: 74 Oenococcus species (e.g. Oenococcus oeni), and SEQ ID NO:

76 in a Bacillus species (e.g. Bacillus coagulans).

Deletion of the ethanol pathway: In one embodiment, the lactic acid bacterium of the invention is characterised by knockout of the endogenous native gene encoding alcohol dehydrogenase (E.C.1.2.1.10) causing a block in the ethanol synthesis pathway in the bacterium. Deletion of the gene encoding an alcohol dehydrogenase enzyme provides a lactic acid bacterium of the invention that is blocked in ethanol production.

For example, where the lactic acid bacterium of the invention belongs to a given genus, the deleted endogenous gene (e.g. adhE) is one encoding a polypeptide having alcohol dehydrogenase activity (E.C.1.2.1.10) in that genus. Preferably the polypeptide having alcohol dehydrogenase activity has at least 70, 72, 74, 76, 78, 80, 82, 84, 86, 88, 90, 92, 94, 96, 98, 99 or 100% amino acid sequence identity to one of the following sequences: SEQ ID NO: 78 in a Lactococcus species (e.g. Lactococcus lactis); SEQ ID NO: 80 in a Lactobacillus species (e.g. Lactobacillus acidophilus); SEQ ID NO: 82 or 84 in a Lactobacillus species (e.g. Lactobacillus casei); SEQ ID NO: 86 in a Lactobacillus species (e.g., Lactobacillus plantarum), SEQ ID NO: 88 in a Leuconostoc species (e.g. Leuconostoc mesenteroides), SEQ ID NO: 90 in a Streptococcus species (e.g. Streptococcus thermophilus), SEQ ID NO: 92 in a Oenococcus species (e.g. Oenococcus oeni), and SEQ ID NO: 94 in a Bacillus species (e.g. Bacillus coagulans).

### Iiv Endogenous Genes Deleted to Block the Endogenous 2,3-butanediol Synthesis Pathway

The lactic acid bacterium of the invention is characterized by knockouts of the endogenous native genes encoding enzymes having α-acetolactate decarboxylase (E.C 4.1.1.5), a diacetyl reductase (EC:1.1.1.303); acetoin reductase (EC:1.1.1.5), and a 2,3-butanediol dehydrogenase ((R,R)-butane-2,3-diol forming; E.C 1.1.1.4/1.1.1.-) activity, thereby causing a block in the 2,3-butanediol synthesis pathway in the bacterium for conversion of α-acetolactate, via D-acetoin or diacetyl, to 2,3-butanediol. Deletion of the endogenous native genes provides a lactic acid bacterium of the invention that is blocked in 2,3-butanediol production. In the case that the lactic acid bacterium of the invention belongs to a given genus, that lacks one or more endogenous native gene encoding one or more polypeptide having α-acetolactate decarboxylase activity (E.C 4.1.1.5), diacetyl reductase (EC:1.1.1.303); acetoin reductase (EC:1.1.1.5), 2,3-butanediol dehydrogenase (E.C 1.1.1.4/1.1.1.-) activity or any combination thereof; the step of deletion of the respective gene in order to produce the bacterium of the invention is not required.

Accordingly the lactic acid bacterium of the invention lacks endogenous native genes that express enzymes having α-acetolactate decarboxylase (E.C 4.1.1.5), diacetyl reductase (EC.1.1.1.303); acetoin reductase (EC:1.1.1.5), and a 2,3-butanediol dehydrogenase ((R,R)-butane-2,3-diol forming; E.C 1.1.1.4/1.1.1.-) activity, either due to the absence of genes encoding and expressing said enzymes in the lactic acid bacterium of the invention, or due to deletion of the respective gene from the genome of the bacterium.

Deletion of an endogenous native gene (e.g. aldB) encoding an α-acetolactate decarboxylase enzyme (E.C 4.1.1.5) provides a lactic acid bacterium of the invention that is blocked in D-acetoin production. For example, where the lactic acid bacterium of the invention belongs to a given genus, the deleted endogenous gene is one encoding a polypeptide having α-acetolactate decarboxylase activity (E.C 4.1.1.5) in that genus.

Preferably the polypeptide having α-acetolactate decarboxylase activity has at least 70, 72, 74, 76, 78, 80, 82, 84, 86, 88, 90, 92, 94, 96, 98, 99 or 100% amino acid sequence identity to one of the following sequences: SEQ ID NO: 96 in a Lactococcus species (e.g. Lactococcus lactis); SEQ ID NO: 98, or 100 in a Lactobacillus species (e.g. Lactobacillus casei, Lactobacillus plantarum), SEQ ID NO: 102 in a Pediococcus species (e.g. Pediococcus pentosaceus), SEQ ID NO: 104 or 106 Leuconostoc species (e.g. Leuconostoc mesenteroides), SEQ ID NO: 108 in a Streptococcus species (e.g. Streptococcus thermophilus), SEQ ID NO: 110 in a Oenococcus species (e.g. Oenococcus oeni), and SEQ ID NO: 112 in a Bacillus species (e.g. Bacillus coagulans).

Deletion of an endogenous native gene (e.g. dar) encoding diacetyl reductase (EC:1.1.1.303) provides a lactic acid bacterium of the invention that is blocked in D-acetoin production. For example, where the lactic acid bacterium of the invention belongs to the genus Lactococcus (e.g. Lactococcus lactis), the deleted gene (dar) encodes a polypeptide having at least 70, 72, 74, 76, 78, 80, 82, 84, 86, 88, 90, 92, 94, 96, 98, 99 or 100% amino acid sequence identity to SEQ ID NO: 114.

Deletion of an endogenous native gene (e.g. ar) encoding an acetoin reductase enzyme (E.C 1.1.1.5) provides a lactic acid bacterium of the invention that is blocked in D-acetoin production. For example, where the lactic acid bacterium of the invention belongs to a given genus, the deleted endogenous gene is one encoding a polypeptide having D-acetoin reductase activity (E.C 1.1.1.5) in that genus. Preferably the polypeptide having D-acetoin reductase activity has at least 70, 72, 74, 76, 78, 80, 82, 84, 86, 88, 90, 92, 94, 96, 98, 99 or 100% amino acid sequence identity to one of the following sequences: SEQ ID NO: 116 in a Lactococcus species (e.g. Lactococcus lactis); SEQ ID NO: 118 in a Pediococcus species (e.g. Pediococcus pentosaceus), SEQ ID NO: 120 or 122 in a Leuconostoc species (e.g. Leuconostoc mesenteroides), SEQ ID NO: 124 or 126 Oenococcus species (e.g. Oenococcus oeni), and SEQ ID NO: 128 in a Bacillus species (e.g. Bacillus coagulans); SEQ ID NO: 214 or 216 in a Lactobacillus species (e.g. Lactobacillus buchneri).

Deletion of a gene (e.g. butAB) encoding 2,3-butanediol dehydrogenase activity (E.C 1.1.1.4/1.1.1.-) provides a lactic acid bacterium of the invention that is blocked in meso-2,3-butanediol production. For example, where the lactic acid bacterium of the invention belongs to the genus Lactococcus (e.g. Lactococcus lactis), the deleted gene (butAB) encodes a polypeptide having at least 70, 72, 74, 76, 78, 80, 82, 84, 86, 88, 90, 92, 94, 96, 98, 99 or 100% amino acid sequence identity to SEQ ID NO: 130.

### Iv Endogenous Genes Deleted to Block the Endogenous NADH Oxidation

The lactic acid bacterium of the invention is characterised by a knockout of the endogenous native gene(s) encoding a water-forming NADH oxidase causing a block in NADH oxidation, and maintenance of reduced NADH levels. Deletion of a gene (e.g. noxE) provides a lactic acid bacterium of the invention that is partially blocked in NADH oxidation.

For example, where the lactic acid bacterium of the invention belongs to a given genus, the deleted endogenous gene is one encoding a polypeptide having water-forming NADH oxidase activity (E.C. 1.6.3.4) in that genus. Preferably the polypeptide having NADH oxidase activity activity has at least 70, 72, 74, 76, 78, 80, 82, 84, 86, 88, 90, 92, 94, 96, 98, 99 or 100% amino acid sequence identity to one of the following sequences: SEQ ID NO: 132 in a Lactococcus species (e.g. Lactococcus lactis); SEQ ID NO: 134 in Lactobacillus casei), SEQ ID NO: 136, 138, 140, 142 and 144 in Lactobacillus plantarum, SEQ ID NO: 146 in a Streptococcus species (e.g. Streptococcus thermophilus), SEQ ID NO: 148, 150 and 152 in a Bacillus species (e.g. Bacillus coagulans).

In the case that the lactic acid bacterium of the invention belongs to a given genus, that lacks an endogenous native gene encoding one or more polypeptide having water-forming NADH oxidase activity (E.C. 1.6.3.4) activity; the step of deletion of the respective gene in order to produce the bacterium of the invention is not required. Accordingly the lactic acid bacterium of the invention lacks endogenous native genes that express an enzyme having water-forming NADH oxidase activity (E.C. 1.6.3.4) activity, either due to the absence of gene encoding said enzyme in the lactic acid bacterium of the invention, or due to deletion of the respective gene from the genome of the bacterium.

### II A Lactic Acid Bacterium Comprising a Pathway for (S,S)-2,3-butanediol Synthesis

The lactic acid bacterium according to the first or second embodiment of the invention, comprising a pathway for synthesis of (S,S)-2,3-butanediol, is a member of a genus of lactic acid bacteria selected from the group consisting of Lactococcus, Lactobacillus, Pediococcus, Leuconostoc, Streptococcus, Oenococcus, and Bacillus. The lactic acid bacterium of the invention may for example be a species of lactic acid bacteria selected from the group consisting of Lactococcus lactis, Lactobacillus acidophilus, Lactobacillus delbrueckii, Lactobacillus casei, Lactobacillus plantarum, Pediococcus pentosaceus, Leuconostoc mesenteroides, Streptococcus thermophilus, Oenococcus oeni and Bacillus coagulans.

### III Methods for Producing a Micro-Organism of the Invention

Integration and self-replicating vectors suitable for cloning and introducing one or more gene encoding one or more a polypeptide having an enzymatic activity associated with (S,S)-2,3-butanediol synthesis in a lactic acid bacterium according to the first or second embodiment of the invention are commercially available and known to those skilled in the art (see, e.g., Sambrook et al., Molecular Cloning: A Laboratory Manual, Second Edition, Cold Spring Harbor Laboratory Press, 1989). Cells of a micro-organism are genetically engineered by the introduction into the cells of heterologous DNA (RNA). Heterologous expression of genes encoding one or more polypeptide having an enzymatic activity associated with (S,S)-2,3-butanediol synthesis in a micro-organism of the invention is demonstrated in the Examples.

A nucleic acid molecule, that encodes one or more polypeptide having an enzymatic activity associated with (S,S)-2,3-butanediol synthesis according to the invention, can be introduced into a cell or cells and optionally integrated into the host cell genome using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

Deletion of endogenous genes in a host lactic acid bacterium to obtain a lactic acid bacterium according to the first or second embodiment of the invention can be achieved by a variety of methods; for example by transformation of the host cell with linear DNA fragments containing a locus for resistance to an antibiotic, or any other gene allowing for rapid phenotypic selection, flanked by sequences homologous to closely spaced regions on the cell chromosome on either side of the gene to be deleted, in combination with the immediate subsequent deletion or inactivation of the recA gene. By selecting for a double-crossover event between the homologous sequences, shown by the antibiotic resistance or other detectable phenotype, a chromosome disruption can be selected for which has effectively deleted an entire gene. Inactivation or deletion of the recA gene prevents recombination or incorporation of extrachromosomal elements from occurring, thereby resulting in a bacterial strain which is useful for screening for functional activity or production of genetically engineered proteins in the absence of specific contaminants. An example describing the deletion of IdhX, IdhB, Idh, pta, adhE, butBA, aldB and noxE from L. lactis given in Example 1, illustrates one method for deleting these genes.

### IV A Method for Producing (S,S)-2,3-butanediol

(S,S)-2,3-butanediol can be produced using a lactic acid bacterium according to the first embodiment of of the invention by introducing the bacterium into a culture medium comprising a carbon source for (S,S)-2,3-butanediol biosynthesis; providing the culture with a source of protoporphyrin IX or iron-containing porphyrin (e.g, hemin and hematin) and incubating under aerobic conditions; and finally recovering the (S,S)-2,3-butanediol produced by the culture, as illustrated in the Examples.

The lactic acid bacterium of the invention will produce (S,S)-2,3-butanediol when supplied with a suitable carbon source including glucose, maltose, galactose, fructose, sucrose, arabinose, xylose, raffinose, mannose, and lactose.

A supply of iron-containing porphyrin, in limited amounts, is essential for the lactic acid bacterium of the invention to grow and produce (S,S)-2,3-butanediol. In a preferred embodiment, the protoporphyrin IX or iron-containing porphyrin is provided to the culture, by addition to the culture medium either prior to and subsequent to the introduction of the lactic acid bacterium into the culture medium. The protoporphyrin IX or iron-containing porphyrin may be added continuously or as a batch addition to the culture during incubation of the culture. For example hemin is preferably added in amounts to provide a final concentration in the liquid culture medium of 0.1-5 μg/ml.

The culture is incubated under aerobic conditions; such conditions being provided by shaking/agitating/stirring the culture under aerobic conditions; or sparging the culture with a source of oxygen. When the lactic acid bacterium of the invention is a strain of Lactococcus lactic, the preferred temperature for cultivation is 30° C.; while the selection of a suitable temperature for growth of lactic acid bacteria of the invention belonging to other Genus lies within the competence of the skilled man.

Where (S,S)-2,3-butanediol is secreted by the lactic acid bacterium of the invention, the (S,S)-2,3-butanediol can be recovered from the growth medium; and where the (S,S)-2,3-butanediol is an intracellular product, it can be recovered from cells of the micro-organism of the invention by permeabilization of cell membranes combined with extraction of the (S,S)-2,3-butanediol, employing standard methods for extraction, including solvent extraction as illustrated in the examples.

### V A Lactic Acid Bacterium for Production of (S,S)-2,3-butanediol Comprising a Diacetyl Insensitive Metabolic Pathway

According to a second embodiment, the present invention provides a genetically modified lactic acid bacterium capable of producing (S,S)-2,3-butanediol stereo specifically from glucose under aerobic conditions; where the bacterium is characterized by three aspects:
1. comprising one transgene: encoding a polypeptide capable of catalyzing the conversion of DA to L-AC (having diacetyl reductase activity; E.C.1.1.1.304) as well catalyzing the conversion of L-AC to L-BD (having L-butanediol dehydrogenase activity; E.C.1.1.1.76);
2. inactivation of a one or more metabolic pathways to enhance metabolic flux from pyruvate for diacetyl, which is the precursor in the pathway for stereospecific synthesis of (S,S)-2,3-butanediol;
3. being capable of producing a high-yield of (S,S)-2,3-butanediol by means of a combination of chemical catalysis (metal catalysis) and metabolic pathways.

The polypeptide having both diacetyl reductase and L-butanediol dehydrogenase activity, expressed by the genetically modified lactic acid bacterium, is characterized by retaining enzymatic activity in the presence of ≧5 mM diacetyl. Preferably the polypeptide retains enzymatic activity in the presence of ≧10 mM, ≧15 mM, ≧20 mM, ≧25 mM; ≧30 mM; ≧35 mM, ≧40 mM; ≧50 mM; ≧55 mM; ≧60 mM or ≧70 mM diacetyl. The amino acid sequence of the polypeptide has at least 70, 72, 74, 76, 78, 80, 82, 84, 86, 88, 90, 92, 94, 96, 98, 99 or 100% sequence identity to the amino acid sequence of the L-butanediol dehydrogenase (SEQ ID NO: 218) (derivable from Enterobacter cloacae bdh gene). Alternatively, the polypeptide having diacetyl reductase and L-butanediol dehydrogenase activity has the amino acid sequence selected from among SEQ ID NO: 220 (derived from Klebsiella pneumoniae budC/dar; ADI56519.1; GI:298108447); SEQ ID NO: 222 (derived from Kluyvera intermedia budC/dar; WP_047370614.1; GI:829955571); and SEQ ID NO: 224 (derived from Enterobacter spp WP_014882919).

Enhanced metabolic flux pyruvate for diacetyl in the genetically modified lactic acid bacterium, is due to reduced activity of one or more enzyme in the pathways leading to the formation of one or more of lactate, ethanol and acetate. The genetically modified lactic acid bacterium according to the second embodiment of the invention is characterized by the inactivation of genes encoding enzymes in the lactate, and optionally also acetate and ethanol pathways (as described in section Iiii). Additionally, stereospecific synthesis of (S,S)-2,3-butanediol is enhanced in the genetically modified lactic acid bacterium according to the second embodiment by deletion of endogenous genes encoding enzymes in the native butandiol synthesis pathway (as described in section Iiv). Additionally, in order to obtain a balanced redox state in the genetically modified lactic acid bacterium according to the second embodiment, the endogenous native gene(s) encoding a water-forming NADH oxidase are deleted (e.g. noxE) (as described in section Iv).

While not being bound by theory, it is hypothesized that the redox status of the genetically modified lactic acid bacterium according to the second embodiment of the invention, is balanced because the NADH generated by glycolysis (2 mol NADH per mol glucose) is then oxidized during the conversion of diacetyl to (S,S)-2,3-butanediol; thereby regenerating NAD (2 mol NADH are consumed per mol (S,S)-2,3-butanediol from diacetyl).

The lactic acid bacterium according to the second embodiment, comprising a diacetyl insensitive metabolic pathway for synthesis of (S,S)-2,3-butanediol, is a member of a genus of lactic acid bacteria as described in Section II.

Methods for producing a genetically modified lactic acid bacterium according to the second embodiment of the invention are described in section III.

### VI A Method for Producing (S,S)-2,3-butanediol Using a Lactic Acid Bacterium Comprising a Diacetyl Insensitive Metabolic Pathway

(S,S)-2,3-butanediol can be produced using a genetically modified lactic acid bacterium according to the second embodiment of the invention by the steps of: introducing the bacterium into a culture medium comprising a carbon source for (S,S)-2,3-butanediol biosynthesis; providing the culture with a source of Fe³⁺; incubating the culture under aerobic conditions; and finally recovering the (S,S)-2,3-butanediol produced by the culture, as illustrated in Example 3. Importantly, a source of protoporphyrin IX or iron-containing porphyrin (e.g, hemin and hematin) is not present in, nor is it added to, the culture medium; i.e. essentially all sources of protoporphyrin IX or iron-containing porphyrin are excluded from the culture medium. This is because the redox balance obtained by culturing the cells in a growth medium comprising a source of Fe³⁺ requires that endogenous hemin-inducible pathways leading to NADH oxidation are not activated.

The lactic acid bacterium of the invention will produce (S,S)-2,3-butanediol, when supplied with a suitable carbon source including glucose, maltose, galactose, fructose, sucrose, arabinose, xylose, raffinose, mannose, and lactose. Preferably the final concentration of the carbon source in the growth medium is equivalent to 0-100 mM glucose.

A supply of Fe³⁺ ions, in an amount of at least 2 mM, is essential for the lactic acid bacterium according to the second embodiment of the invention to grow. Surprisingly, when provided with a sufficient supply of Fe³⁺ ions, cells of the lactic acid bacterium are able to produce (S,S)-2,3-butanediol with an efficiency that is close to the maximum possible. For example, yields of 0.89 mol (S,S)-2,3-butanediol/mol glucose (corresponding to 89% of the maximum theoretical yield) was demonstrated using a genetically modified Lactococcus lactis bacterium according to the second embodiment of the invention (see Example 3).

The source of Fe³⁺ ions is provided to the culture, by addition to the culture medium either prior to and subsequent to the introduction of the lactic acid bacterium into the culture medium. The supply of Fe³⁺ ions may be added continuously or as a batch addition to the culture during incubation of the culture. For example Fe³⁺ ions are preferably added in amounts to provide a final concentration in the liquid culture medium in the range of 3 to 30 mM; more preferably, in the range of 3 to 25 mM; for example in the range of 5 to 20 mM.

The culture is incubated under aerobic conditions; such conditions being provided by shaking/agitating/stirring the culture under aerobic conditions; or sparging the culture with a source of oxygen. When the lactic acid bacterium of the invention is a strain of Lactococcus lactic, the preferred temperature for cultivation is 30° C.; while the selection of a suitable temperature for growth of lactic acid bacteria of the invention belonging to other Genus lies within the competence of the skilled man.

Where (S,S)-2,3-butanediol is secreted by the lactic acid bacterium of the invention, the (S,S)-2,3-butanediol can be recovered from the growth medium; and where the (S,S)-2,3-butanediol is an intracellular product, it can be recovered from cells of the micro-organism of the invention by permeabilization of cell membranes combined with extraction of the (S,S)-2,3-butanediol, employing standard methods for extraction, including solvent extraction as illustrated in the examples.

### VII A Method of Detecting (S,S)-2,3-butanediol Produced

Methods for detecting and quantifying (S,S)-2,3-butanediol produced by a micro-organism of the invention include high performance liquid chromatography (HPLC) combined with Refractive Index detection to identify and quantify (S,S)-2,3-butanediol and its biosynthetic precursors, relative to a set of standards for each step of their biosynthetic pathway, as described herein, as illustrated in the examples.

### EXAMPLES

### Example 1 Genetic Modification of a Lactococcus lactis Strain for Production of S, S-2,3-butanediol

The genetic modifications required to produce a Lactococcus lactis strain that is capable of producing (S,S)-2,3-butanediol from diacetyl and to efficiently direct the flux towards this compound include the inactivation of all alternative product pathways, as described below.

### 1.1 Host Strains and Plasmids

The plasmid-free strain Lactococcus lactis subsp. cremoris MG1363 (Gasson, 1983) or derivatives thereof were used as the parent strain for the genetic construction of a strain capable of producing S,S-2,3-butanediol. E. coli strain ABLE-C (E. coli C lac(LacZ-)[Kanr McrA-McrCB-McrF-Mrr-HsdR (rk-mk-)][F′proAB lacIqZΔM15 Tn10(Tetr)]) (Stratagene) was used for cloning purposes. The plasmid pCS1966 (Solem et al., 2008), was used for the purpose of deleting various genes in L. lactis. The plasmid pCI372 (Hayes et al., 1990) was used for expressing the synthetic dar-bdh operon.

### 1.2 DNA Techniques

All manipulations were performed according to Sambrook et al., (1989). PCR primers used can be seen in TABLE 1. PfuX7 polymerase (Norøholm, 2010) was used for PCR applications. Chromosomal DNA from L. lactis was isolated using the method described for E. coli with the modification that cells were treated with 20 μg of lysozyme per ml for 2 hours before lysis. Cells of E. coli were transformed using electroporation. Cells of L. lactis were made electrocompetent by growth in GM17 medium containing 1% glycine and transformed by electroporation as previously described by Holo and Nes (1989).

The plasmid vector pCS1966 (Solem et al., 2008) was used for deleting genes in L. lactis. Plasmids employed for deleting chromosomal genes were prepared by PCR amplifying approximately 800 base pairs (bp) regions upstream and downstream of the L. lactis chromosomal region to be deleted using the PCR primers and chromosomal DNA isolated from L. lactis. The primers used for amplifying the upstream and downstream regions are indicated in TABLE 1 as “geneX ups.” and geneX dwn”. The amplified fragments and the plasmid, pCS1966, were then digested with the respective restriction enzymes indicated in the primer table, prior to inserting the fragment into the plasmid. The resulting plasmids were transformed into the parent strain individually and gene deletion was performed as described by Solem C, et al. (2008). Specifically, the plasmids were transformed into the strains via electroporation, and the strains comprising the plasmids integrated into the chromosome were selected for on M17 plates supplemented with glucose and erythromycin. Afterwards, the transformants were purified and plated on SA glucose plates supplemented with 5-fluoroorotate, thereby selecting for strains in which the plasmid had been lost by homologous recombination. The successful deletions were verified by PCR (Solem et al., 2008).

### 1.3 Deleting Genes from the Lactococcus lactis subsp. cremoris

The following genes were deleted from the Lactococcus lactis subsp. cremoris parent strain IdhX, IdhB, Idh, pta, adhE, butBA, aldB and noxE. The genes were deleted using gene deletion plasmids derived from pCS1966 designated as: pCS4026 (IdhX), pCS4020 (IdhB), pCS4104 (Idh), pCS4230 (pta), pCS4273 (adhE), pCS4491 (butBA), pCS4495 (aldB) and pCS4256 (noxE), constructed as described above (Example 1.2).

Deletion of the genes from the Lactococcus lactis subsp. cremoris parent strain was verified by PCR amplification of the respective gene using primers 774/777 (IdhX), 769/771 (IdhB), 788/789 (Idh), 880/881(pta), 929/930 (adhE), 977/979 (butBA), 1117/1119 (aldB), 887/890 (noxE).

TABLE 1
Strains and plasmids

| Designation | Genotype or description | Reference |
|---|---|---|
| L. lactis strains | | |
| CS4363 | MG1363 Δ³ldh Δpta ΔadhE | Solem et al., 2013 |
| CS4311 | MG1363 Δ³ldh Δpta ΔadhE pCS4268 | This work |
| CS4502 | *MG1363 Δ³ldh Δpta ΔadhE ΔbutBA pCS4268 | This work |
| CS4525 | *MG1363 Δ³ldh Δpta ΔadhE ΔbutBA ΔaldB pCS4268 | This work |
| CS4554 | MG1363 ΔldhX ΔldhB Δpta ΔadhE ΔbutBA ΔaldB ΔnoxE | This work |
| | pCS4268 | |
| CS4562 | MG1363 Δ³ldh ΔadhE ΔbutBA ΔaldB ΔnoxE pCS4564 | This work |
| CS4616 | MG1363 Δ³ldh Δpta ΔadhE ΔbutBA ΔaldB ΔnoxE pCS4564 | This work |
| CS4616m | MG1363 Δ³ldh Δpta ΔadhE ΔbutBA ΔaldB ΔnoxE | This work |
| CS4634 | MG1363 pCS4634 (pCI372::SP-budC-bdh) | This work |
| CS4701 | MG1363 Δ³ldh Δpta ΔadhE ΔbutBA ΔaldB ΔnoxE pCS4634 | This work |
| CS4701m | MG1363 Δ³ldh Δpta ΔadhE ΔbutBA ΔaldB ΔnoxE pJM001 | This work |
| Plasmids | | |
| pG⁺host8 | E. coli/L. lactis shuttle vector, Tet^{R}, thermosensitive | Maguin et al., 1996 |
| | replicon | |
| pCS4268 | pG⁺host8::SP-idh (L. lactis) | This work |
| pCS4564 | pG⁺host8::SP-idhA (E. coli) | This work |
| pCI372 | E. coli/L. lactis shuttle vector, Cam^{R} | Hayes et al., 1990 |
| pCS4518 | pCI372::gusA | This work |
| pCS4634 | pCS4518::SP-budC-bdh | This work |
| pJM001 | pTD6::FP-bdh | This work |

*Indicates that the chromosomal ldh may have reverted to wild-type by recombination with pCS4268; Δ³ldh = Δldh ΔldhX ΔldhB; SP signifies Synthetic Promoter. FP means fixed promoter, ATAGATTAGTTTATTCTTGACACTACAAGCTAAATGTGGTATAATCCCATAGA [SEQ ID NO: 225] (-35 and -10 underlined).

The strain containing the three lactate dehydrogenase deletions (Idh, IdhB, IdhX) was named CS4099 or MG1363Δ3Idh. CS4234 was derived from CS4099, by additionally the deleting a phosphotransacetylase gene, pta. The CS4234 strain deleted for the three Idh genes had poor growth properties; so to facilitate growth of the strain and its subsequent genetic modifications, the CS4234 strain was transformed with a plasmid with a thermosensitive replicon carrying L. lactis Idh expressed from a synthetic promoter (SP), to give strain CS4268. The plasmid was prepared as follows: an SP-Idh fragment was amplified from L. lactis using primers 710/926, was digested with XbaI/XhoI and inserted into pG+host8 plasmid (Maguin et al., 1996) digested with the same enzymes, and the ligated plasmid was then introduced into the CS4234 strain. CS4311 was derived from strain

CS4268 by deletion of adhE; CS4502 was derived from strain CS4311 by deletion of butBA, and CS4525 was derived from strain CS4502 by deletion of deleted a/dB. CS4554 was derived from strain CS4525 by deletion of noxE, but in this strain Idh was found to have reverted to wild-type (Idh) due to a recombination event between the deleted Idh locus and the intact Idh gene on the pG+host8 plasmid. CS4562 was derived from strain CS4554 lacking the pG+host8 plasmid, but substituted by another pG+host8 plasmid carrying an E. coli IdhA (pCS4564). The plasmid pCS4564 was constructed in the following manner: SP-IdhA was amplified from E. coli using 1130/1131, digested with XhoI/XbaI and inserted into pG+host8 digested with the same enzymes. The chromosomal Idh was then deleted from CS4562 thus giving rise to CS4615 (MG1363 ΔIdh ΔIdhX ΔIdhB Δpta ΔadhE ΔbutBA ΔaldB ΔnoxE pCS4564).

### 1.4 Introducing Codon-Optimized Diacetyl Reductase (dar) and Butanediol Dehydrogenase (bdh) into Lactococcus lactis subsp. cremoris Strain CS4615

A synthetic codon-optimized operon consisting of budC/dar gene sequence (encoding diacetyl/acetoin reductase, accession no. AF098800) from Klebsiella pneumonia tranascriptionally fused to a budC/bdh gene sequence (encoding acetoin reductase, accession no. AB009078) from Brevibacterium saccharolyticum was ordered from GenScript. The gene sequence, encoding an Aldolase leader sequence, (from L. lactis): 1-30; fused to a first gene sequence, budC: 31-798 (without stop codon, 801 with stop codon TAA), fused to a gene sequence, encoding the gapB leader (from L. lactis): 802-828, fused to a second gene bdh: 829-1602 (1605 with stop codon TAA included) and finally fused to the groEL2 transcriptional terminator (from L. lactis): 1606-1642.

Plasmid pCS4518 was constructed by ligating PCR amplified pCI372 (primers 1112/1113) with gusA PCR amplified from E. coli MG1655 (primers 991/992) that was treated with T4 PNK. Plasmid pCS4518 was then amplified using primers 1113/991, and joined using T4 DNA ligase to SP-dar-bdh amplified using 893/975, (treated with T4 polynucleotide kinase) and then introduced in E. coli TOP10, used as a host. Plasmids isolated from the pool of transformants were introduced into the deletion strain MG1363 and a clone (CS4634) showing the highest expression of the dar-bdh operon was selected and its plasmid (pCS4634) was isolated. pCS4634 was then introduced into the non-integrating plasmid

CS4615 (MG1363 ΔIdhΔIdhX ΔIdhB Δpta ΔadhE ΔbutBA ΔaldB ΔnoxE pCS4564); where after the plasmid pCS4564 was lost by incubation at 36° C. The resulting strain was CS4701.

TABLE 2

| Primer | | |
|---|---|---|
| name | Primer use | Primer sequence (5′→3′) |
| 43 (T3) | Verify insert | AATTAACCCTCACTAAAGGG [SEQ ID NO: 153] |
| | in pCS1966 | |
| 603 | Verify insert | ATCAACCTTTGATACAAGGTTG [SEQ ID NO: 154] |
| | in pCS1966 | |
| 710 | SP-ldh, XbaI | CTAGTCTAGANNNNAGTTTATTCTTGACANNNNNNNNNNN |
| | | NNTGRTATAATNNNNAAGTAATAAAATATTCGGAGGAATTTTG |
| | | AAATGGCTGATAAACAACGTAAG [SEQ ID NO: 155] |
| 768 | ldhB ups., | AATTCCTGCAGCATATTAAATAATGAACAAGTCATTC [SEQ ID |
| | PstI | NO: 156] |
| 769 | ldhB ups., | TAGTGGATCCTGGTAAATCCAAACACAACAAC [SEQ ID NO: |
| | BamHI | 157] |
| 770 | ldhB dwn., | AATTCCTGCAGTAATTTCCAGCTCTTACAATAAC [SEQ ID NO: |
| | PstI | 158] |
| 771 | ldhB dwn., | GACCTCGAGTCAGAAACTTTCTTTACCAGAG [SEQ ID NO: 159] |
| | XhoI | |
| 772 | pCS1966, | GCGGGGATCCACTAGTTCTAG [SEQ ID NO: 160] |
| | BamHI | |
| 773 | pCS1966, | ATACCGTCGACCTCGAG [SEQ ID NO: 161] |
| | XhoI | |
| 774 | ldhX ups., | TAGTGGATCCCTGTTTCAGGTCTTGGATAG [SEQ ID NO: 162] |
| | BamHI | |
| 775 | ldhX ups., | CCGATGAATTCTCATTAGCACGTTTAACAAGAG [SEQ ID NO: |
| | EcoRI | 163] |
| 776 | ldhX dwn., | CCGATGAATTCATCAGCGTAGTCTGCTGC [SEQ ID NO: 164] |
| | EcoRI | |
| 777 | ldhX dwn., | CGGGGTACCATTTAATCCTAAAGTCGTTATTAC [SEQ ID NO: |
| | KpnI | 165] |
| 785 | ldh ups., | CCGATGAATTCTTAAGTCAAGACAACGAGGTC [SEQ ID NO: |
| | EcoRI | 166] |
| 786 | ldh dwn, | CCGATGAATTCGACCTTGTTGAAAAAAATCTTC [SEQ ID NO: |
| | EcoRI | 167] |
| 787 | ldh ups., | TAGTGGATCCGTACAATGGCTACTGTTAAC [SEQ ID NO: 168] |
| | BamHI | |
| 788 | ldh dwn., | GACCTCGAGGATGAACAGACTTTTTTATTATAG [SEQ ID NO: |
| | XhoI | 169] |
| 789 | Verify ldh | AAAACCAGGTGAAACTCGTC [SEQ ID NO: 170] |
| | deletion | |
| 791 | adhB rev, | TCGGACTGCAGTTAAAATGCTGATAAAAACAATTCTTC +SEQ |
| | PstI | ID NO: 171] |
| 827 | pCS1966, | ATACCGTCGACCTCGAG [SEQ ID NO: 172] |
| | BamHI | |
| 828 | pCS1966, | CGATAAGCTTGATATCGAATTC [SEQ ID NO: 173] |
| | XhoI | |
| 830 | adhB fwd, | CCGATGAATTCTATAAGGAGAATTAGAATGGCAAGTAGTACAT |
| | EcoRI | TTT ATATTC [SEQ ID NO: 174] |
| 878 | pta ups., | ATCCCTCGGTTACAAGTTTCU [SEQ ID NO: 175] |
| | USER | |
| 879 | pta dwn., | AGAAACTTGTAACCGAGGGAUAATAATAGATTGAAATTCTGTC |
| | USER | AG [SEQ ID NO: 176] |
| 880 | pta ups., | ATTCGATATCAAGCTTATCGAUCAAAAATTGTGGTAGAATATA |
| | USER | TAG [SEQ ID NO: 177] |
| 881 | pta dwn., | AGGTCGACGGTATCGATAAUCCTAGTTCAATTGATGTGAC |
| | USER | [SEQ ID NO: 178] |
| 882 | pCS1966, | ATCGATAAGCTTGATATCGAAU [SEQ ID NO: 179] |
| | USER | |
| 883 | pCS1966, | ATTATCGATACCGTCGACCU [SEQ ID NO: 180] |
| | USER | |
| 887 | noxE ups, | ATTCGATATCAAGCTTATCGAUATTTAAAAATGATTGCAACAT |
| | USER | ATAAC [SEQ ID NO: 181] |
| 888 | noxE ups, | ATAGGTCTCCTTTAAATGTAAAAU [SEQ ID NO: 182] |
| | USER | |
| 889 | noxE dwn, | ATTTTACATTTAAAGGAGACCTAUTAGAAATCTATCTGCTTGA |
| | USER | TAG [SEQ ID NO: 183] |
| 890 | noxE dwn, | AGGTCGACGGTATCGATAACGUCTTCACCGTCCATTTTGAC |
| | USER | [SEQ ID NO: 184] |
| 891 | pTD6, USER | ACAGATTAAAGGTTGACCAGTAU [SEQ ID NO: 185] |
| 892 | pTD6, USER | ACCAATTCTGTGTTGCGCAU [SEQ ID NO: 186] |
| 893 | SP-dar-bdh, | ATGCGCAACACAGAATTGGUGGCCNNNNNAGTTTATTCTTGAC |
| | fwd. | ANNNNNNNNNNNNNNTGRTATAATNNNNAAGTAATAAAATAT |
| | | TCGGAGGAAT [SEQ ID NO: 187] |
| 894 | adhB rev., | ATACTGGTCAACCTTTAATCTGUTTAAAATGCTGATAAAAACA |
| | USER | ATTCTT [SEQ ID NO: 188] |
| 920 | pCS1966, | ATAAGCTUGATATCGAATTCCT [SEQ ID NO: 189] |
| | USER | |
| 921 | pCS1966, | ATTCCCTTUAGTGAGGGTTAAT [SEQ ID NO: 190] |
| | USER | |
| 926 | ldh rev, XhoI | TCGACCTCGAGTTTTTTATTTTTAGTTTTTAACTGCAG [SEQ ID |
| | | NO: 191] |
| 927 | adhE ups., | ATGTGTACGUTCTCCTTTGTG [SEQ ID NO: 192] |
| | USER | |
| 928 | adhE dwn., | ACGTACACAUATTATAGTATTTGGAACCGAAC [SEQ ID NO: |
| | USER | 193] |
| 929 | adhE ups., | AAGCTTAUGGTCGTCTTGTTACTTGTG [SEQ ID NO: 194] |
| | USER | |
| 930 | adhE dwn., | AAAGGGAAUTCTGCCGGAGCTATATATG [SEQ ID NO: 195] |
| | USER | |
| 975 | dar-bdh rev. | TTAATTATACAACATTCCTCCATC [SEQ ID NO: 196] |
| 976 | butBA ups., | AATTCCTGCAGATCTATACCTACTTGACCAGC [SEQ ID NO: 197] |
| | PstI | |
| 977 | butBA ups., | TAGTGGATCCGAGTATTCGCAAACCTTCAG [SEQ ID NO: 198] |
| | BamHI | |
| 978 | butBA dwn., | AATTCCTGCAGAATAAATGAATGAGGTAAGGTCTA [SEQ ID |
| | PstI | NO: 199] |
| 979 | butBA dwn., | GACCTCGAGTTTAAGAGATAAAAGGTTAATTGTG [SEQ ID NO: |
| | XhoI | 200] |
| 991 | gusA | GAATCGGTACCAATAAAATATTCGGAGGAATTTTGAAATGTTA |
| | MG1655 | CGTCCTGTAGAAAC [SEQ ID NO: 201] |
| 992 | gusA | GGACCGTACGTTAAAAAATAAAAAAGAACCCACTCGGGTTCTT |
| | MG1655 | TTTTTTATTGTTTGCCTCCCTGCTG [SEQ ID NO: 202] |
| 1057 | aldB ups., | TAGTGGATCCCTTAATTGCTGGAATCACTG [SEQ ID NO: 203] |
| | BamHI | |
| 1058 | aldB ups., | AATTCCTGCAGATGATATTTCTCTTTTCTATCTCA [SEQ ID NO: |
| | PstI | 204] |
| 1059 | aldB dwn., | AATTCCTGCAGAATTGCTTAAATTTCTTTAGCTAC [SEQ ID NO: |
| | PstI | 205] |
| 1060 | aldB dwn., | TCGACCTCGAGTTAGACGCTCGGGATAAAG [SEQ ID NO: 206] |
| | XhoI | |
| 1112 | pCI372 | GCAACAACGTGCGCAAAC [SEQ ID NO: 207] |
| 1113 | pCI372 | CTGCAGGTCGACTCTAG [SEQ ID NO: 208] |
| 1117 | aldB fwd. | AATATTTTAGGACCCAATGATG [SEQ ID NO: 209] |
| 1119 | aldB rev | CGAGCTGGAAAGCTTTTATC [SEQ ID NO: 210] |
| 1130 | SP-IdhA E. | CTAGTCTAGAGCNNAGTTTATTCTTGACANNNNNNNNNN |
| | coli, XbaI | NNNNTGRTATAATNNNNAAGTAATAAAATATTCGGAGGAATT |
| | | TTGAAATGAAACTCGCCGTTTATAG [SEQ ID NO: 211] |
| 1131 | ldhA rev, | TCGACCTCGAGAAGAATAGAGGATGAAAGGTC [SEQ ID NO: |
| | XhoI | 212] |

### 1.5 Properties of the Genetically Engineered Strain

A strain of Lactococcus lactis subsp. cremoris from which the lactate dehydrogenases (Idh, IdhB, IdhX), phosphotransacetylase (pta), and alcohol dehydrogenase (adhE) have been inactivated by deletion of their genes is only able to grow aerobically. The main fermentation products of this strain are D-acetoin, diacetyl and pyruvate. The formation of 2,3-butanediol from diacetyl consumes the two NADH formed in glycolysis. In order to obtain a high yield of 2,3-butanediol, it is essential to eliminate alternative NADH consuming reactions. Aerobic growth conditions are needed for the non-enzymatic conversion of α-acetolactate into diacetyl, but unfortunately if oxygen is present NADH oxidase activity consumes a large proportion of the NADH. The main source of NADH oxidase activity in L. lactis can be attributed to NoxE (>95%), which is a water-forming NADH oxidase. The noxE gene was there for deleted in the final strain, CS4701. In addition, the α-acetolactate decarboxylase gene (aldB) and butBA operon were deleted to avoid formation of D-acetoin, which can only be converted into meso- or (R,R)-2,3-butanediol, and interference from the native butanediol dehydrogenases. To enable conversion of diacetyl into (S,S)-2,3-butanediol two 2,3-butanediol dehydrogenases were expressed as an operon from synthetic promoters in a plasmid (pCS4634): ButC from Klebsiella pneumonia which has a high specific activity towards diacetyl and an L-butanediol dehydrogenase from Brevibacterium saccharolyticum. When the chromosomally encoded lactate dehydrogenases and phosphotransacetylase were inactivated the result was a large decline in the specific growth rate and this reduced transformation efficiency and thereby the succeeding manipulations. For this reason we introduced plasmids with a thermosensitive replicon expressing lactate dehydrogenase activity. These plasmids also allowed for efficient regeneration of NAD+and thereby anaerobic growth and were removed in the final strain CS4701. Strain CS4701 (MG1363 ΔIdh ΔIdhX ΔIdhB Δpta ΔadhE ΔbutBA ΔaldB ΔnoxE pCS4634) was found to be unable to grow unless acetoin or hemin was added to the medium.

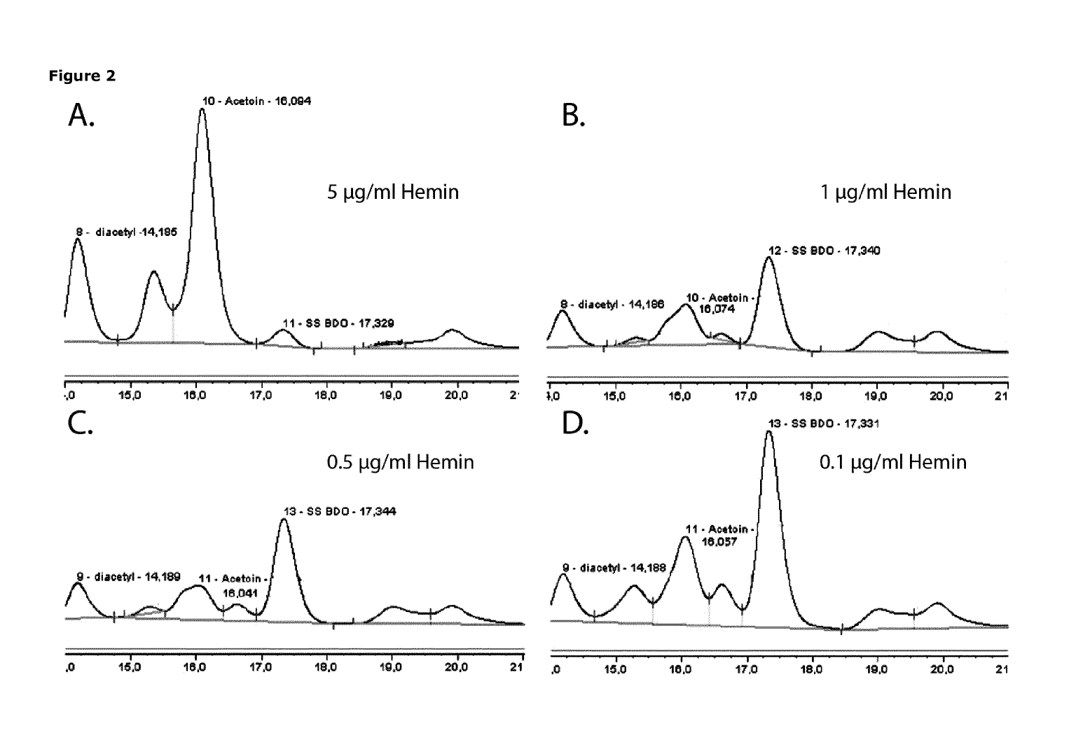

Product formation for strain CS4701 depends on hemin concentration. In principle strain CS4701 should be able to grow and produce (S,S)-2,3-butanediol as the sole fermentation product. This was however not found to be the case and the strain was only able to grow in the presence of hemin, which restores respiration in L. lactis. First CS4701 was grown in M17 medium with 2% glucose and 5 μg/ml hemin but HPLC analysis of the fermentation only revealed small amounts of (S,S)-2,3-butanediol (FIG. 2A). By decreasing the amount of hemin added, however, increasing amounts of (S,S)-2,3-butanediol was formed (FIG. 2B-D). In addition to diacetyl, L-acetoin and (S,S)-2,3-butanediol large amounts of pyruvate were found in the fermentation broth (Table 3).

### Example 2 Production of Stereo-Specific S,S-2,3b

The strain CS4701 derived from Lactococcus lactis subsp. cremoris was grown in M17 medium (supplied by Oxoid; Terzaghi & Sandine, 1975) supplemented with 2% glucose as carbon source, under aerobic conditions at 30° C. In theory, although strain CS4701 was expected to grow and produce (S,S)-2,3-butanediol as the sole fermentation product under these conditions, this was not found to be the case. However, growth could be restored by cultivation in the presence of hemin, which was found to restore respiration in L. lactis. Cultures were grown for 4 days (repeated 2 times) in the presence of different amounts of hemin, where product formation was found to depend on hemin concentration. When strain CS4701 was grown in M17 medium with 2% glucose and 5 μg/ml hemin only revealed small amounts of (S,S)-2,3-butanediol were detected by HPLC analysis of the fermentation (FIG. 2A). By decreasing the amount of hemin added, however, increasing amounts of (S,S)-2,3-butanediol was formed (FIG. 2 B-D). In addition to diacetyl, acetoin and 2,3-butanediol large amounts of pyruvate were found in the fermentation broth (Table 3).

TABLE 3

| | Glu- | Pyru- | Dia- | Ace- | S,S- | Yield | Total |
|---|---|---|---|---|---|---|---|
| | cose | vate | cetyl | toin | BDO | (mol/mol | (glucose |
| | (mM) | (mM) | (mM) | (mM) | (mM) | glucose) | eq) |
| 5 μg/ml | 16.4 | 37.2 | 16.5 | 44.8 | 1.8 | 0.02 | 100 |
| hemin | | | | | | | |
| 1 μg/ml | 40.5 | 60.4 | 5.7 | 11.1 | 6.3 | 0.11 | 100 |
| hemin | | | | | | | |
| 0.5 μg/ml | 42.7 | 58.1 | 5.3 | 6.1 | 7.2 | 0.13 | 100 |
| hemin | | | | | | | |
| 0.1 μg/ml | 24.0 | 44.8 | 6.4 | 9.3 | 13.6 | 0.18 | 100 |
| hemin | | | | | | | |

HPLC was used to determine product composition, using a HPLC column HPX-87H, as described by the manufacturer (http://info.bio-ad.com/rs/bioradlaboratoriesinc/images/Bulletin_6333_Aminex%20HPLC.pdf).

### Example 3 Genetically Modified Lactococcus lactis Strain Comprising a Diacetyl Insensitive L-butanediol Dehydrogenase for Production of S, S-2,3-butanediol

The genetic modifications required to produce a Lactococcus lactis strain that is capable of producing (S,S)-2,3-butanediol from diacetyl and to efficiently direct the flux towards this compound include a set of genetic modifications that result in the inactivation of one or more alternative product pathways, as described in Example 1. Further genetic modification to produce a strain that is capable of producing S,S-2,3-butanediol with high efficiency, is described below.

### 3.1 Introducing Codon-Optimized Butanediol Dehydrogenase (bdh) into a Diacetyl-Producing Lactococcus lactis subsp. cremoris Strain

A synthetic gene [SEQ ID No. 217] encoding a diacetyl-insensitive L-butanediol dehydrogenase (EC 1.1.1.76) [SEQ ID No. 218] was cloned and expressed in the deletion strain CS4616m (MG1363 Δ³Idh Δpta ΔadhE ΔbutBA ΔaldB ΔnoxE), derived from the plasmid-free parent strain Lactococcus lactis subsp. cremoris MG1363 (Gasson, 1983). The expressed butanediol dehydrogenase corresponds to the butanediol dehydrogenase from Enterobacter cloacae having Acc. no. JN035909. The synthetic gene, codon-optimized for expression in L. lactis, was first cloned into the plasmid pTD6 (Solem et al., 2013) and operably linked to a high strength promoter having the nucleotide sequence: ATAGATTAGTTTATTCTTGACACTACAAGCTAAATGTGGTATAATCCCATAGAAGGT [SEQ ID No. 225] (Jensen et al., 1998), resulting in the plasmid pJM001. The plasmid pJM001 was transformed into deletion strain CS4616m thereby yielding the final strain CS4701m (MG1363 Δ³Idh Δpta ΔadhE ΔbutBA ΔaldB ΔnoxE pJM001).

### 3.2 Production of Stereo-Specific S,S-2,3-butanediol

CS4701m was grown in 500 ml conical flasks with 50 ml M17 broth supplemented with glucose at 30° C. and 200 rpm under aerobic conditions. Samples were collected periodically for determining cell density, glucose, α-acetolactate, acetoin and butanediol isomer concentrations. Growth of strain CS4701m was found to be dependent on a supply of hemin. However, in the absence of hemin, growth could be restored by the addition of Fe³⁺ in the beginning of growth phase, detected as an increase in cell density of the culture after 24 h cultivation, measured as OD_{600 nm} (Table 4).

TABLE 4
S-BDO production under different concentrations of Fe³⁺

| | | | | | | Yield (mol |
|---|---|---|---|---|---|---|
| | | Initial | Con- | Ace- | | S-BDO/mol |
| Fe³⁺ | | Glu | sumed | toin | S-BDO | consumed |
| (mM) | OD₆₀₀ | (mM) | Glu¹ | (mM) | (mM) | glucose) |
| 0 | 0.2 | 45.00 | 0.08 | ND | 1.72 | 0.45 |
| 3 | 1.71 | 45.00 | 1.00 | 4.76 | 25.84 | 0.57 |
| 5 | 2.01 | 45.00 | 1.00 | 4.08 | 28.05 | 0.62 |
| 8 | 1.86 | 45.00 | 0.98 | 1.88 | 32.90 | 0.74 |
| 10 | 1.61 | 45.00 | 0.93 | ND | 37.39 | 0.89 |
| 15 | 1.61 | 45.00 | 0.77 | ND | 30.40 | 0.87 |
| 20 | 1.26 | 45.00 | 0.61 | ND | 24.65 | 0.89 |
| 30 | 0.67 | 45.00 | 0.20 | ND | 5.73 | 0.63 |

¹consumed glucose percentage:
ND: Not detectable

TABLE 5
S-BDO production under different concentrations of Fe³⁺

| | | | | | Yield (mol |
|---|---|---|---|---|---|
| | Initial | Con- | Ace- | | S-BDO/mol |
| Fe³⁺ | Glu | sumed | toin | S-BDO | consumed |
| (mM) | (mM) | Glu¹ | (mM) | (mM) | glucose) |
| 0 | 95.00 | 0.04 | 0.00 | 1.42 | 0.43 |
| 5 | 95.00 | 0.89 | 2.27 | 74.00 | 0.8 |
| 10 | 95.00 | 0.91 | 3.40 | 70.61 | 0.81 |
| 15 | 95.00 | 0.53 | 2.05 | 43.58 | 0.87 |
| 20 | 95.00 | 0.41 | 3.40 | 35.00 | 0.89 |
| 30 | 95.00 | 0.19 | 1.80 | 11.11 | 0.61 |

¹consumed glucose percentage

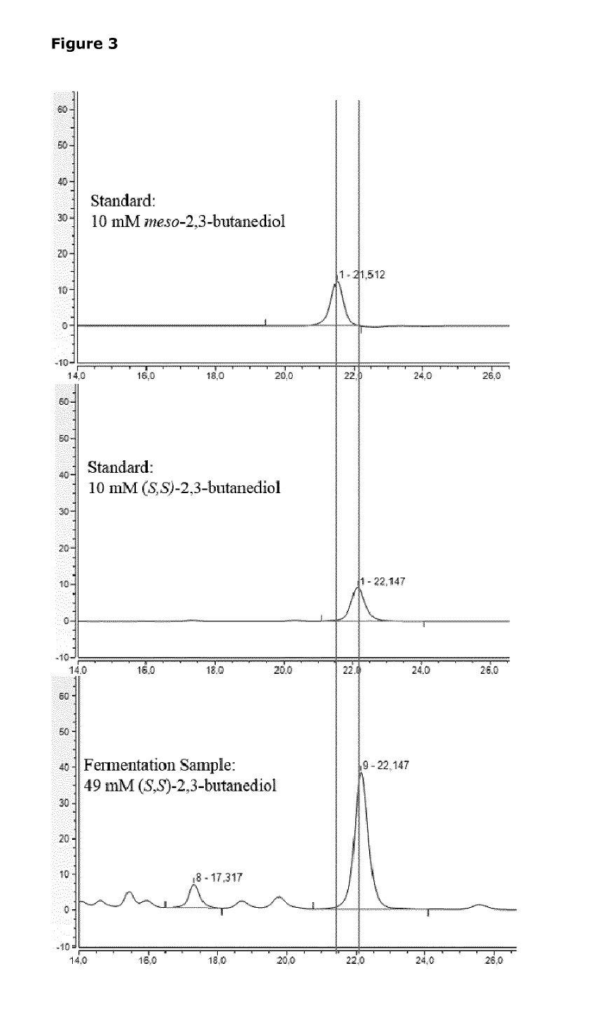

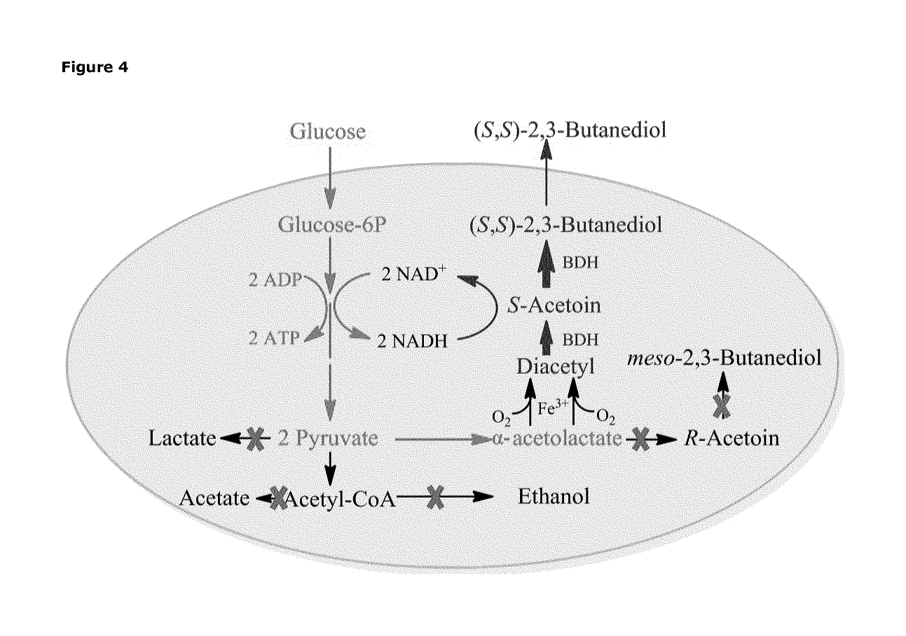

When cells of the strain were grown in the presence of 10 mM Fe³⁺ and glucose at an initial concentration of 45 mM, the levels of S,S-2,3-butanediol (S-BDO) produced by the cells reached a maximum of 37.4 mM S-BDO (3.4 g/I) after 24 h fermentation. The calculated S-BDO yield was 0.89 mol/mol glucose (corresponding to 89% of the maximum theoretical yield). When the initial glucose concentration was set to 95 mM (Table 5), the S-BDO titer increased to 74 mM (6.7 g/l) with a S-BDO yield of 0.8 mol/mol glucose (corresponding to 80% of maximum theoretical yield). The optimal Fe³⁺ concentration supporting S-BDO lay within a range; where concentrations of ≧30mM were less advantageous for S-BDO formation. The butanediol formed by the cells was enantiomerically pure S-BDO (FIG. 3). As seen in FIG. 4, the reductive synthesis of S-BDO serves to recycle the NADH produced by glycolysis and thereby maintains a balanced ratio of the cofactors NAD⁺ and NADH. Fe³⁺, provided in the growth medium, plays an essential role in facilitating co-factor recycling between glycolysis and S-BDO production in the cells, by catalyzing the conversion of α-acetolactate synthase (ALS) into diacetyl by non-enzymatic oxidative decarboxylation, which is one of the rate-limiting steps in diacetyl formation.

### REFERENCES

1. Gasson, M J. 1983. Plasmid complements of Streptococcus lactis NCDO 712 and other lactic streptococci after protoplast-induced curing. J. Bacteriol. 154:1-9
2. Hayes, F., Daly, C., and G. F. Fitzgerald. 1990. Identification of the Minimal Replicon of Lactococcus lactis subsp. lactis UC317 Plasmid pCI305. Appl. Environ Microbiol. 56:202-209
3. Holo H, Nes I F. 1989. High-frequency transformation, by electroporation, of Lactococcus lactis subsp. cremoris grown with glycine in osmotically stabilized media. Appl. Environ. Microbiol. 55:3119-3123
4. Maguin, E., Prevost, H., Ehrlich, S. D. and Gruss, A. (1996) Efficient insertional mutagenesis in lactococci and other Gram-positive bacteria. J Bacteriol 178, 931-935.
5. Nørholm M H H. 2010. A mutant Pfu DNA polymerase designed for advanced uracil-excision DNA engineering. BMC Biotechnol. 10:21
6. Sambrook, J. E. F. Fritsch, and Maniatis. 1989. Molecular cloning: a laboratory manual, 2^{nd} ed. Cold Spring Harbor Laboratory Press, Cold Spring Harbor, N. Y.
7. Solem C, Defoor E, Jensen P R, Martinussen J. 2008. Plasmid pCS1966, a new selection/counterselection tool for lactic acid bacterium strain construction based on the oroP gene, encoding an orotate transporter from Lactococcus lactis. Appl. Environ. Microbiol. 74:4772-4775
8. Terzaghi B E, Sandine W E. 1975. Improved medium for lactic streptococci and their bacteriophages. Appl. Microbiol. 29:807-813
9. Solem, C., Dehli, T. & Jensen, P. R. Rewiring Lactococcus lactis for ethanol production. Appl. Environ. Microbiol. 79, 2512-2518 (2013).
10. Jensen, P. R. & Hammer, K. The sequence of spacers between the consensus sequences modulates the strength of prokaryotic promoters. Appl. Environ. Microbiol. 64:82-87 (1998).

1. A genetically modified lactic acid bacterium for production of S,S-2,3-butanediol, wherein said microorganism comprises one or more transgene encoding one or more polypeptide, wherein the one or more polypeptide has an enzymatic activity of:
a. a diacetyl reductase (E.C. 1.1.1.304) and
b. a L-butanediol dehydrogenase (E.C. 1.1.1.76) and wherein the genome of said lactic acid bacterium is deleted for genes or lacks genes encoding polypeptides having an enzymatic activity of:
c. lactate dehydrogenase (E.C1.1.1.27 or E.C.1.1.1.28)
d. α-acetolactate decarboxylase (E.C4.1.1.5)
e. diacetyl reductase (E.C.1.1.1.303)
f. butanediol dehydrogenase (E.C. 1.1.1.4)
g. acetoin reductase (EC: 1.1.1.5) and
h. NADH oxidase (E.C. 1.6.3.4).

2. A genetically modified lactic acid bacterium according to claim 1, wherein the genome of said lactic acid bacterium is additionally deleted for genes encoding polypeptides having an enzymatic activity of:
i. phosphotransacetylase (E.C.2.3.1.8) and
j. alcohol dehydrogenase (E.C. 1.2.1.10).

3. A genetically modified lactic acid bacterium according to claim 1, wherein the lactic acid bacteria belongs to a genus selected from the group consisting of Lactococcus, Lactobacillus, Pediococcus, Leuconostoc, Streptococcus, Oenococcus, and Bacillus.

4. A genetically modified lactic acid bacterium according to claim 1, wherein said microorganism comprises one transgene encoding one polypeptide, wherein the one polypeptide has an enzymatic activity of a diacetyl reductase (E.C.1.1.1.304) and a L-butanediol dehydrogenase (E.C. 1.1.1.76) and is capable of converting diacetyl to S,S-2,3-butanediol.

5. A genetically modified lactic acid bacterium according to claim 4, wherein the amino acid sequence of the polypeptide capable of converting diacetyl to S,S-2,3-butanediol has 80% to 100% sequence identity to an amino acid sequence selected from among SEQ ID NO:
218, 220, 222, and 224.

6. A genetically modified lactic acid bacterium according to claim 1, wherein said microorganism comprises one transgene encoding one polypeptide having an enzymatic activity of a diacetyl reductase (E.C. 1.1.1.304) and one transgene encoding one polypeptide having an enzymatic activity of a L-butanediol dehydrogenase (E.C. 1.1.1.76).

7. A genetically modified lactic acid bacterium according to claim 1, wherein the amino acid sequence of the polypeptide having diacetyl reductase (E.C.1.1.1.304) activity has at least 80% sequence identity to an amino acid sequence selected from among SEQ ID NO: 2, 4, 6, 8 and 10.

8. A genetically modified lactic acid bacterium according to claim 1, wherein the amino acid sequence of the polypeptide having L-butanediol dehydrogenase activity (E.C. 1.1.1.76) has at least 80% sequence identity to an amino acid sequence selected from among SEQ ID NO: 12, 14 and 16.

9. A genetically modified lactic acid bacterium according to claim 1, wherein the amino acid sequence of the polypeptide having lactate dehydrogenase activity has at least 80% sequence identity to an amino acid sequence selected from among SEQ ID NO: 18, 20, 22, 24, 26, 28, 30, 32, 34, 36, 38, 40, 42, 44, 46, 48, 50 and

52.

10. A genetically modified lactic acid bacterium according to claim 1, wherein the amino acid sequence of the polypeptide having phosphotransacetylase activity has at least 80% sequence identity to an amino acid sequence selected from among SEQ ID NO: 58, 60, 62, 64, 66, 68, 70, 72, 74 and 76, and wherein the amino acid sequence of the polypeptide having alcohol dehydrogenase activity has at least 80% sequence identity to an amino acid sequence selected from among SEQ ID NO: 78, 80, 82, 84, 86, 88, 90, 92 and 94.

11. A genetically modified lactic acid bacterium according to claim 1, wherein the amino acid sequence of the polypeptide having α-acetolactate decarboxylase activity has at least 80% sequence identity to an amino acid sequence selected from among SEQ ID NO: 96, 98, 100, 102, 104, 106, 108, 110 and 112.

12. A genetically modified lactic acid bacterium according to claim 1, wherein the amino acid sequence of the polypeptide having diacetyl reductase (E.C. 1.1.1.303) activity has at least 80% sequence identity to SEQ ID NO: 114.

13. A genetically modified lactic acid bacterium according to claim 1, wherein the amino acid sequence of the polypeptide having acetoin reductase activity has at least 80% sequence identity to SEQ ID NO: 116, 118, 120, 122 124, 126 and 128, and wherein the amino acid sequence of the polypeptide having butanediol dehydrogenase (E.C. 1.1.1.4) activity has at least 80% sequence identity to SEQ ID NO: 130.

14. A genetically modified lactic acid bacterium according to claim 1, wherein the amino acid sequence of the polypeptide having a NADH oxidase activity has at least 80% sequence identity to an amino acid sequence selected from among SEQ ID NO: 132, 134, 136, 138, 140, 142, 144, 146, 148, 150 and 152.

15. A method for the production of S,S-2,3-butanediol, comprising the steps of:
a. introducing a genetically modified lactic acid bacterium according to claim 1 into a growth medium to produce a culture,
b. providing a source of protoporphyrin IX or iron-containing porphyrin, or providing a source of Fe³⁺ ions;
c. providing aerobic culture conditions,
d. recovering S,S-2,3-butanediol produced by said culture, and optionally
e. isolating the recovered S,S-2,3-butanediol.

16. A method for the production of S,S-2,3-butanediol according to claim 15, wherein the source of iron-containing porphyrin is hemin or hematin.

17. A method for the production of S,S-2,3-butanediol according to claim 15, wherein the concentration of hemin is 0.1-5 μg/ml growth medium.

18. A method for the production of S,S-2,3-butanediol according to claim 15, wherein the Fe³⁺ ion concentration of the growth medium is at least 2 mM; and wherein a source of protoporphyrin IX or iron-containing porphyrin is excluded.

19. Use of a genetically modified lactic acid bacterium according to claim 1 for production of acetoin and S,S-2,3-butanediol.
